(19) United States
(12) Patent Application Publication
(10) Pub. No.: US 2022/0268478 A1
(43) Pub. Date: Aug. 25, 2022

(54) HOT/COLD SENSATION ESTIMATING DEVICE, METHOD, AND PROGRAM

(71) Applicant: KABUSHIKI KAISHA TOSHIBA, Tokyo (JP)

(72) Inventors: Takashi SUDO, Fuchu Tokyo (JP); Izumi FUKUNAGA, Tokyo (JP)

(73) Assignee: KABUSHIKI KAISHA TOSHIBA, Tokyo (JP)

(21) Appl. No.: 17/461,709

(22) Filed: Aug. 30, 2021

(30) Foreign Application Priority Data
Feb. 24, 2021 (JP) 2021-027482

(51) Int. Cl. F24F 11/64; A61B 5/00; A61B 5/08; A61B 5/01; A61B 5/05; G05B 19/042

(52) U.S. Cl. CPC F24F 11/64; A61B 5/4035; A61B 5/4005; A61B 5/4266; A61B 5/0816; A61B 5/0077; A61B 5/015; A61B 5/05; A61B 5/01; A61B 5/4064; G05B 19/042; F24F 2110/10

(57) ABSTRACT

A hot/cold sensation estimating device according to one embodiment includes a sensing unit and an estimating unit. The sensing unit is configured to sense a behavioral thermoregulatory reaction and an autonomic thermoregulatory reaction of living matter against an ambient environment based on sensor data acquired through a sensor for sensing vital activities of the living matter. The estimating unit is configured to estimate a hot/cold sensation sensed by the living matter based on a sensing result of the behavioral thermoregulatory reaction and a sensing result of the autonomic thermoregulatory reaction.

### CROSS-REFERENCE TO RELATED APPLICATIONS

This application is based upon and claims the benefit of priority from Japanese Patent Application No. 2021-027482, filed Feb. 24, 2021, the entire contents of which are incorporated herein by reference.

### FIELD

Embodiments described herein relate generally to a hot/cold sensation estimating device, method, and program.

### BACKGROUND

As objective indexes to estimate a hot/cold sensation sensed by living matter such as a human, a predicted mean vote (PMV) and a standard effective temperature (SET) are known. These indexes are used to execute air conditioning control of, for example, an air conditioning apparatus.

### BRIEF DESCRIPTION OF THE DRAWINGS

FIG. 1 is a diagram showing a configuration example of an air conditioner including a hot/cold sensation estimating device according to a first embodiment.

FIG. 2 is a flowchart showing an example of a procedure of a hot/cold sensation estimating process performed by the hot/cold sense estimating device according to the first embodiment.

FIG. 3 is a diagram showing a configuration example of an air conditioner including a hot/cold sensation estimating device according to a first modification of the first embodiment.

FIG. 4 is a flowchart showing an example of a procedure of a hot/cold sense estimating process performed by a hot/cold sensation estimating device according to the first modification of the first embodiment.

FIG. 5 is a diagram showing a configuration example of an air conditioner including a hot/cold sensation estimating device according to a second embodiment.

FIG. 6 is a flowchart showing an example of a procedure of a hot/cold sensation estimating process performed by a hot/cold sense estimating device according to the second embodiment.

### DETAILED DESCRIPTION

A hot/cold sensation estimating device according to one embodiment includes a sensing unit and an estimating unit. The sensing unit is configured to sense a behavioral thermoregulatory reaction and an autonomic thermoregulatory reaction of living matter against an ambient environment based on sensor data acquired through a sensor for sensing vital activities of the living matter. The estimating unit is configured to estimate a hot/cold sensation sensed by the living matter based on a sensing result of the behavioral thermoregulatory reaction and a sensing result of the autonomic thermoregulatory reaction.

Embodiments of a hot/cold sensation estimating device, method, and program will be described in detail with reference to the drawings. In the following description, structural elements having substantially the same functions and configurations will be denoted by the same reference symbols, and repeat descriptions of such elements will be given only where necessary.

### First Embodiment

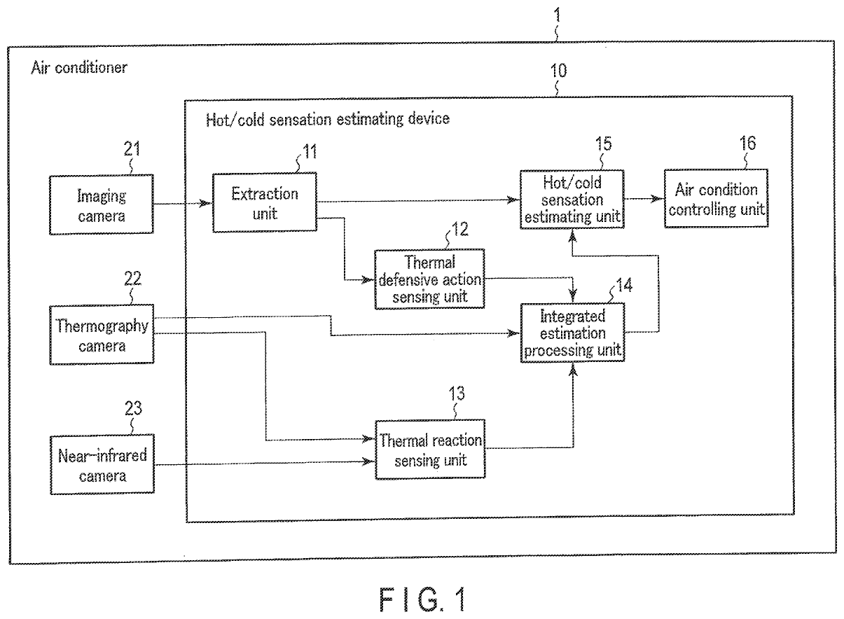

FIG. 1 is a diagram showing a configuration of an air conditioner 1 including a hot/cold sensation estimating device 10 according to a first embodiment. The air conditioner 1 is an air conditioning apparatus that performs air-conditioning control using a controller. The air conditioner 1 is located in a home or an office. The air conditioner 1 includes a louver, a compressor, a fan, etc., to send controlled air. The air conditioner 1 further includes a hot/cold sensation estimating device 10 and a sensor that senses vital activities of living matter. The hot/cold sensation estimating device 10 estimates a hot/cold sensation sensed by the living matter from sensor data acquired through the sensor. The hot/cold sensation is a thermal comfort sensed by living matter such as a human. The hot/cold sensation is expressed by scalar values of a continuous range from +3 to −3 corresponding to subjective expressions, such as +3: hot, +2: warm, +1: slightly warm, 0: neutral, −1: slightly cool, −2: cool, and −3: cold. As a matter of course, the hot/cold sensation may be expressed by scalar values of a continuous range from +4 to −4 corresponding to subjective expressions, such as +4: very hot, +3: hot, +2: warm, +1: slightly warm, 0: neutral, −1: slightly cool, −2: cool, −3: cold, and −4: very cold. The hot/cold sensation estimating device 10 estimates a thermoregulatory ability of the living matter, and uses it to estimate a hot/cold sensation. Furthermore, the hot/cold sensation estimating device 10 senses a behavioral thermoregulatory reaction and an autonomic thermoregulatory reaction of living matter with respect to the ambient environment, and uses them to estimate a thermoregulatory ability and a hot/cold sensation.

The behavioral thermoregulatory reaction includes a thermal defensive action. The thermal defensive action is a defensive reaction against heat that is generated due to an involuntary vital reaction. The autonomic thermoregulatory reaction includes a thermal reaction. The thermal reaction includes a heat dissipation reaction that dissipates heat to the ambient environment, and a heat production reaction that causes heat to flow in from the ambient environment. The hot/cold sensation may be referred to as a “hot/cold thermal sensation”.

The sensor is a non-contact sensor that senses vital activities of living matter without contacting the living matter. In the embodiments, the air conditioner 1 includes an imaging camera 21, a thermography camera 22, and a near-infrared camera 23. The imaging camera 21 may be a general camera for generating a two-dimensional image or a depth camera for generating a three-dimensional image including depth information. Instead of the near-infrared camera 23, a radar (microwaves, millimeter waves, etc.), LiDAR (Light Detection and Racing), or ToF (Time of Flight (infrared light type, light pulse type, ultrasonic pulse type, etc.)) may be used.

The hot/cold sensation estimating device 10 includes a processing circuit configured to control the overall hot/cold sensation estimating device 10 and a storage medium (memory). The processing circuit is a processor configured to execute functions of an extraction unit 11, a thermal defensive action sensing unit 12, a thermal reaction sensing unit 13, an integrated estimation processing unit 14, a hot/cold sensation estimating unit 15, and an air condition controlling unit 16 by invoking and executing programs in the storage medium. The processing circuit is formed of an integrated circuit including a central processing unit (CPU), an application specific integrated circuit (ASIC), a field programmable gate array (FPGA), or the like. The processor may be formed of either one integrated circuit or a plurality of integrated circuits.

The storage medium stores processing programs executed by the processor, and parameters, tables, and the like for use in computation by the processor. The storage medium is a storage device, such as a hard disk drive (HDD), a solid state drive (SSD), or an integrated circuit, etc. which stores various types of information. The storage device is not limited to the HDD, SSD, etc., but also a portable storage medium, such as a compact disc (CD), a digital versatile disc (DVD), or a flash memory, or a driver that writes and reads various types of information in and from, for example, a semiconductor memory, such as a flash memory or a random access memory (RAM).

The processing circuit that implements the functions of the extraction unit 11, the thermal defensive action sensing unit 12, and the thermal reaction sensing unit 13 is an example of a sensing unit. The processing circuit that realizes the functions of the integrated estimation processing unit 14 and the hot/cold sensation estimating unit 15 is an example of an estimating unit. The functions of the extraction unit 11, the thermal defensive action sensing unit 12, the thermal reaction sensing unit 13, the integrated estimation processing unit 14, the hot/cold sensation estimating unit 15, and the air condition controlling unit 16 may be realized by a single processing circuit. Alternatively, a processing circuit may be constituted by a combination of a plurality of independent processors which respectively realize the functions by executing the respective programs. Alternatively, the functions of the extraction unit 11, the thermal defensive action sensing unit 12, the thermal reaction sensing unit 13, the integrated estimation processing unit 14, the hot/cold sensation estimating unit 15, and the air condition controlling unit 16 may be respectively implemented as hardware circuits.

The extraction unit 11 acquires sensor data obtained by sensing vital activities of living matter from the sensor. The sensor data is, for example, image data imaged by the imaging camera 21. The extraction unit 11 extracts living matter as a target for estimation of a hot/cold sensation. The living matter is a homeothermic animal having an ability to regulate its body temperature. The living matter is, for example, a human. The living matter may be a pet, such as a dog, a cat, a bird, or a mouse, or even livestock, such as a horse, a cow, or a pig. The extraction unit 11 extracts a person of living matter as a target for estimation of a hot/cold sensation by performing a human extraction process for an image captured by the imaging camera 21. The extraction unit 11 also estimates a body part of the extracted person by performing a human tracking process, such as joint model fitting, of this extracted person, and detects a motion from this extracted person. The extraction unit 11 outputs this motion of the extracted person to the thermal defensive action sensing unit 12 and the hot/cold sensation estimating unit 15. In the case of using a depth camera instead of the imaging camera 21, a motion in a depth direction of the image can also be detected.

The thermal defensive action sensing unit 12 senses a behavioral thermoregulatory reaction of the extracted living matter based on the sensor data. Specifically, the thermal defensive action sensing unit 12 senses a thermal defensive action of the living matter extracted by the extraction unit 11, or senses whether the living matter is capable of taking a thermal defensive action, based on the sensor data acquired through the sensor and extraction data acquired from the extraction unit 11. The thermal defensive action includes an action to raise the body temperature when it is cold (hereinafter referred to as “an action expressing coldness”) or an action to lower the body temperature when it is hot (hereinafter referred to as “an action expressing hotness”). The thermal defensive action sensing unit 12 senses an action expressing coldness or an action expressing hotness using a detection result of a motion of the living matter in the extraction unit 11.

An action expressing coldness is an action to generate heat inside the body or absorb external heat into the body. The action expressing coldness can be, for example, shivering, rubbing, reducing an exposed area of skin, putting on clothes, taking up a body position to reduce skin surface area, shrinking, moving to a hot place, intaking hot food or drink, making contact with a hot object, such as a Kotatsu, a hot-water bag, a portable body warmer, etc.

An action expressing hotness is an action to release heat from the body. The action expressing hotness is, for example, taking up a body position to increase a surface area of skin, increasing an exposed area of skin, removing clothes, taking a spread-eagled position, moving to a cold place, intaking cold food or drink, making contact with a cold object, bathing in water, fanning oneself, etc.

The thermal reaction sensing unit 13 senses an autonomic thermoregulatory reaction of the extracted living matter, or senses whether the living matter is capable of taking an autonomic thermoregulatory reaction, based on the sensor data. Specifically, the thermal reaction sensing unit 13 senses a thermal reaction of the living matter extracted by the extraction unit 11 based on the sensor data acquired through the sensor. The thermal reaction includes a heat dissipation reaction that dissipates heat to the ambient environment, and a heat production reaction that causes heat to flow in from the ambient environment.

The heat dissipation reaction is a vital reaction that occurs unconsciously in hot conditions. The heat dissipation reaction is, for example, sweating, such as thermal sweating or gustatory sweating, or evaporative heat dissipation caused by breathing. The heat dissipation reaction also includes an autonomous vital reaction, such as an increase in the degree of opening the mouth while inhaling and exhaling, an increase in the breathing rate, and performance of mouth breathing. The heat dissipation reaction includes blood vessel dilation. When the blood flow rate is increased by blood vessel dilation, the skin temperature rises and the amount of heat dissipation increases.

The heat production reaction is a vital reaction that occurs unconsciously in cold conditions. The heat production reaction includes, for example, inflow of heat through the mouth by breathing. The heat production reaction also includes an autonomous vital reaction, such as a decrease in the degree of opening the mouth while inhaling and exhaling, a decrease in the breathing rate, and performance of nasal breathing. The heat production reaction includes a fat combustion reaction due to muscle tension, trembling, an involuntary motion of a skeletal muscle, chills, goose bumps, a shaking knee motion, a rubbing motion, or the like. The heat production reaction includes a decrease of the blood volume due to urination. When the blood volume decreases, the amount of water in the body decreases and the amount of heat produced increases. The heat production reaction includes secretion of a hormone or adrenaline. When a hormone or adrenaline is secreted, a metabolic reaction or heat production in the body is promoted. The heat production reaction includes contraction of the blood vessels. When the blood flow rate is decreased by contraction of the blood vessels, the skin temperature lowers and the amount of heat dissipation decreases. The heat production reaction includes a decrease of the pulse rate. When the pulse rate decreases, the blood flow rate decreases and the amount of heat dissipation decreases.

The frequency of breath, the heat amount of exhalation, and the heat amount of inhalation are calculated, for example, by means of a thermography image acquired from the thermography camera 22. The amount of sweating and the amount of moisture on the skin surface are calculated, for example, based on a temporal change of the image or by means of a machine-learned model, using a radar, or ToF, a thermography image acquired from the thermography camera 22 or a near-infrared image acquired from the near-infrared camera 23. The contraction or dilation of the blood vessels is sensed, for example, by means of a face image acquired from the imaging camera 21. The pulse rate is calculated, for example, by estimating a pulse wave from a change in luminance value of a green component in a region in which many capillary blood vessels are exposed on the skin surface, such as the forehead and cheek, using the face image acquired from the imaging camera 21, and counting the number of peaks in the pulse wave per unit time. Secretion of a hormone or adrenaline is sensed by, for example, analyzing a change in pulse rate.

The integrated estimation processing unit 14 performs an integrated estimation process with respect to a sensing result of a thermal defensive action and a sensing result of a heat dissipation reaction. In the integrated estimation process, the integrated estimation processing unit 14 first calculates an amount of heat dissipated to the environment through the heat dissipation reaction (hereinafter referred to as “the amount of heat dissipation”) and an amount of heat inflow from the environment (hereinafter referred to as “the amount of heat inflow”) based on the sensing result of the heat dissipation reaction. The sum of these heat amounts is defined as an amount of heat obtained by thermal reaction.

The amount of heat [J] is calculated by an amount of heat per unit time and unit area (heat flux)×cross-sectional area [m²]×time [s]. Therefore, the amount of heat dissipation is calculated by, for example, the following equation (1):

Amount of heat dissipation=(Frequency of breath×Heat amount of exhalation×Mouth size+Heat amount of skin moisture evaporation×Efficiency×Surface area)×Time (1)

In the equation (1), “frequency of breath” represents the number of breaths per unit time. “Heat amount of exhalation” represents the amount of heat dissipated by one breath per unit area. “Mouth size” represents the cross-sectional area of the mouth which opens when breathing. “Heat amount of skin moisture evaporation” represents the amount of heat evaporated per unit area due to evaporation of moisture from the skin surface. “Efficiency” represents the value of frequency of occurrence of evaporation from the skin surface per unit time. “Surface area” represents the area of a region of the skin surface in which moisture is sensed. “Time” represents the length of time of the sensed heat dissipation reaction. However, since the heat amounts for exhalation and skin moisture evaporation are the amounts of heat due to dissipation and evaporation, they are thus dependent on the temperature or humidity of the ambient air. For example, if the ambient temperature is lower than the body temperature, the heat amount of exhalation and the heat amount of skin moisture evaporation are approximately zero, and therefore, the amount of heat dissipation is approximately zero. When the ambient temperature is higher than the body temperature, the heat amount of exhalation and the heat amount of skin moisture evaporation take positive values, and therefore the amount of heat dissipation takes a positive value.

The amount of hair on the head or the body may be estimated from an image captured by the imaging camera 21, and a calculation result of the amount of heat dissipation from the skin may be corrected based on these hair amounts. A hair style may be estimated from the image captured by the imaging camera 21, for example, whether the hair on the head covers the ears, or the hair is long, short, or thin. Then, the calculation result of the amount of heat dissipation from the skin may be corrected in accordance with a variation of the hair style. Thus, the amount of heat dissipation or the hot/cold sensation can be estimated in consideration of the personal physical sensation.

The amount of heat inflow is calculated by, for example, the following equation (2):

Amount of heat inflow=(Frequency of breath×Heat amount of inhalation×Mouth size+Heat amount of conduction on skin×Conductivity×Surface area)×Time (2)

In the equation (2), “Frequency of breath” represents the number of breaths per unit time. “Heat amount of inhalation” represents the amount of heat inflow in one breath per unit area. “Mouth size” represents the cross-sectional area of the mouth which opens when breathing. “Heat amount of conduction on skin” represents the amount of heat conducted from the ambient air or ambient heat source to the skin surface via radiation or conduction per unit area. “Conductivity” represents the value of frequency of conduction of heat to the skin surface via radiation or conduction per unit time. However, the heat amount of inhalation and the heat amount of conduction on skin depends on the temperature or humidity of the ambient air. When the ambient temperature is lower than the body temperature, the heat amounts for inhalation and conduction on skin take negative values, and therefore the amount of heat inflow takes a negative value. A negative value of the amount of heat inflow means that heat is drawn from the body. When the ambient temperature is higher than the body temperature, the heat amounts for inhalation and conduction on skin take positive values, and therefore the amount of heat inflow takes a positive value.

Next, the integrated estimation processing unit 14 integrates the sensing result of a thermal defensive action and the sensing result of a thermal reaction by changing the sensing result of the thermal reaction using the detection result of the thermal defensive action. At this time, based on the sensing result of the thermal defensive action, the integrated estimation processing unit 14 first calculates the amount of heat released from the body through the thermal defensive action and the amount of heat caused to flow into the body through the thermal defensive action. Next, the integrated estimation processing unit 14 changes a parameter of the amount of heat dissipation using the sensing result of the thermal defensive action, and uses the altered parameter as an amount of heat released from the body. The integrated estimation processing unit 14 also changes a parameter of the amount of heat inflow using the sensing result of the thermal defensive action, and uses the altered parameter as an amount of heat flow into the body.

An example of a method for changing the sensing result of the thermal reaction using the sensing result of the thermal defensive action will be explained. The integrated estimation processing unit 14 first senses the surroundings of the living matter through the thermography camera 22, and extracts the temperature of the ambient air (ambient temperature). Alternatively, the integrated estimation processing unit 14 may extract the temperature of the ambient air (ambient temperature) using an environment sensor provided in the air conditioner 1, such as a thermometer. For example, when a thermal defensive reaction of the action expressing hotness occurs because the body temperature or the skin surface temperature is higher than the ambient temperature, the surface area in the equation for calculating the amount of heat dissipation is increased by the removal of clothes or the adoption of a spread-eagled position to increase the exposed area of skin, or the efficiency in the equation for calculating the amount of heat dissipation is increased by the act of fanning or of moving to a cold place. Otherwise, the heat amount of conduction on the skin in the amount of heat inflow is rendered a negative value by the act of moving to a cold place, intaking cold food or drink, making contact with a cold object, bathing in water, etc. On the contrary, when a heat production reaction of the action expressing coldness occurs because the body temperature or the skin surface temperature is lower than the ambient temperature, the surface area in the equation for calculating the amount of heat dissipation is decreased by the wearing of clothes, the adoption of a body position to reduce the surface area of the skin and decrease the exposed area of skin, or the efficiency in the equation of calculating the amount of heat dissipation is decreased by the act of shivering or rubbing. Otherwise, the heat amount of conduction on the skin in the amount of heat inflow is increased to become a large negative value by the act of moving to a hot place, intaking hot food or drink, making contact with a hot object, such as a Kotatsu, a hot-water bag, a portable body warmer, etc.

The hot/cold sensation estimating unit 15 estimates a hot/cold sensation sensed by the living matter based on the sensing result of the behavioral thermoregulatory reaction, including the thermal defensive action, and the sensing result of the autonomic thermoregulatory reaction, including the thermal reaction. An index indicative of a hot/cold sensation is an objective evaluation value simulating a subjective evaluation of the hot/cold sensation using a detection value obtained by the sensor.

The index indicative of a hot/cold sensation is, for example, a predicted mean vote (hereinafter referred to as the “PMV”). The PMV is an index estimating a heat comfort based on physical consideration from human sensory amounts. The PMV is an index which takes into account two factors on the side of a human body (a metabolic equivalent [METs] and an amount of clothing [clo]) in addition to four factors on the side of an environment that determine the hot/cold sensation (an air temperature (dry-bulb temperature) [° C.], a humidity [%], a wind velocity [m/s], and thermal radiation [° C.]). The PMV of the value 0 represents a thermally neutral state, and the values −3 to +3 of the PMV represent the heat comfort of a human. The PMV is approximately calculated by, for example, the following equation (3), where L is a thermal load of a human body [W/m²] and M is a metabolic amount [W/m²]. Each of the thermal load L and the metabolic amount M is a value that varies depending on any of the air temperature, the humidity, the wind velocity, the thermal radiation, the metabolic equivalent, and the amount of clothing.

PMV={0.303·exp(−0.036·M)+0.028}L (3)

The air temperature, the humidity, the wind velocity, and the thermal radiation are acquired, for example, through the environment sensor provided in the air conditioner 1, and stored in the storage medium. The environment sensor is, for example, a dry-bulb thermometer, a wet-bulb thermometer, a hygrometer, a wind velocity sensor, or the like. The wind velocity may be calculated from a wind direction, an air volume, a wind velocity, a wind strength, or the like controlled by the air conditioner 1. The thermal radiation is an average radiation temperature, and calculated from a dry-bulb temperature, a wet-bulb temperature, and a wind velocity. The air temperature may be calculated by using a thermography image acquired from the thermography camera 22. Since the metabolic equivalent of 1 [METs] corresponds to 58.2 [W/m²], the metabolic amount may be calculated from the metabolic equivalent [METs] by detecting a motion of a person extracted by the extraction unit 11 from an image captured by the imaging camera 21 or a thermography image acquired from the thermography camera 22, and estimating a specific movement of the motion. As a correspondence between a metabolic equivalent and a specific movement, an average value of a general human may be used: for example, the metabolic equivalent is 1.0 [METs] in a seated position at rest, 1.2 [METs] in a standing position at rest, and 2.0 [METs] in walking at a low speed indoors. The amount of clothing may be calculated by estimating clothes from the image captured by the imaging camera 21, for example, a half-sleeve shirt or a long-sleeve shirt, half pants or long pants, etc. As a correspondence between clothes and an amount of clothing, an average value of a general human may be used: for example, the amount of clothing is 0.3 [clo] for a half-sleeve shirt plus half pants, 0.4 [clo] for a long-sleeve shirt plus long pants, 1.0 [clo] for a jacket and long pants, etc. The average values of the metabolic equivalent and the amount of clothing are stored in, for example, the storage medium.

As a method for estimating an index indicative of hot/cold sensation represented by a scalar value ranging from −3 to +3, a method utilizing the combination of a fluctuation and a gradient of a peripheral skin temperature may be utilized. For example, assuming that a skin surface temperature at the tip of a nose is the peripheral skin temperature, the hot/cold sensation of a person may be estimated by detecting this person's tip of the nose from an image captured by the imaging camera 21 or a thermography image acquired from the thermography camera 22, extracting the skin surface temperature at the tip of the nose from the thermography image acquired from the thermography camera 22, and calculating the fluctuation and the gradient of the skin surface temperature at the tip of the nose. The body part at which the peripheral skin temperature is extracted may be an ear lobe, a fingertip, a toe, or the like. At such a peripheral body part, the skin is probably exposed to air even when the person wears clothing, and the angular field of the non-contact sensor probably covers and detects the site. Therefore, the hot/cold sensation can be easily estimated at that site.

At rest, when the temperature lowers, the pulse rate decreases due to the lack of blood, and when the temperature rises, the pulse rate increases. Therefore, a value obtained by calculation using the pulse rate, an interval between peaks of the pulse wave (Peak to Peak interval, PPI), or pulse fluctuation may be utilized as an index indicative of the hot/cold sensation. In this case, a pulse wave is extracted from a change in luminance value of a green component in a region in which many capillary blood vessels are exposed on the skin surface, such as the forehead and cheek, or a peripheral body part (a nose tip, an ear lobe, a fingertip, a toe, etc.), using the face image acquired from the imaging camera 21, and the peak interval (PPI) is calculated to calculate its coefficients of variance, namely, a standard deviation of the NN intervals (SNND), a percentage difference between adjacent NN intervals greater than x microseconds (pNNx), a root mean square of successive differences (RMSSD), a coefficient of variation of R-R interval (CVRR), a low-frequency (LF), a high-frequency (HF), LF/HF, etc. Since it is assumed that each of these coefficients and a hot/cold sensation have a negative correlation, the hot/cold sensation may be estimated by using a regression formula calculated in advance, for example, a multiplication with a negative coefficient.

As an index indicative of a hot/cold sensation, a standard effective temperature (hereinafter referred to as SET) may also be used. A general index indicative of a hot/cold sensation, such as the PMV or SET, is calculated using a general value, for example, an average value, of the amount of clothing or the metabolic equivalent. In other words, the index, such as the PMV or SET, is an index applicable to everyone, with a personal thermoregulatory ability not reflected within.

When the PMV is used as an index indicative of a hot/cold sensation, the hot/cold sensation estimating unit 15 calculates the PMV as an index indicative of the hot/cold sensation sensed by the living matter, and corrects the calculated value of the PMV based on a result of integrating sensing results of both the thermal defensive action and heat dissipation reaction. At that time, the hot/cold sensation estimating unit 15 corrects the calculated value of the PMV using the sensing result of the thermal reaction altered using the sensing result of the thermal defensive action. In the following, a case of using the PMV expressed by a scalar value in a consecutive range as the index indicative of the hot/cold sensation will be mainly described. In the description using the PMV, the PMV may be replaced with another index expressed by a scalar value in a continuous range and estimated by a method which bears no relation to amount of clothing.

For example, when an action expressing coldness is detected as the thermal defensive action, the sensing result of the thermal reaction is changed to increase the amount of heat inflow. The hot/cold sensation estimating unit 15 corrects the index indicative of the hot/cold sensation in accordance with the altered amount of heat inflow. For example, in the case of using the PMV as the index indicative of the hot/cold sensation, the hot/cold sensation estimating unit 15 increases the value of the PMV in accordance with the altered amount of heat inflow. On the other hand, when an action expressing hotness is detected as the thermal defensive action, the sensing result of the thermal reaction is changed to increase the amount of heat dissipation. The hot/cold sensation estimating unit 15 corrects the index indicative of the hot/cold sensation in accordance with the altered amount of heat dissipation. For example, in the case of using the PMV as the index indicative of the hot/cold sensation, the hot/cold sensation estimating unit 15 decreases the value of the PMV in accordance with the altered amount of heat dissipation.

When a heat dissipation reaction to allow heat to flow out to the environment is sensed, the hot/cold sensation estimating unit 15 corrects the index indicative the hot/cold sensation in accordance with the amount of heat dissipation. For example, in the case of using the PMV as the index indicative of the hot/cold sensation, the hot/cold sensation estimating unit 15 decreases the value of the PMV in accordance with the amount of heat dissipation. On the other hand, when a heat production reaction to allow heat to flow in from the environment is sensed, the hot/cold sensation estimating unit 15 corrects the index indicative the hot/cold sensation in accordance with the amount of heat inflow. For example, in the case of using the PMV as the index indicative of the hot/cold sensation, the hot/cold sensation estimating unit 15 increases the value of the PMV in accordance with the amount of heat inflow.

For estimation by the hot/cold sensation estimating unit 15, a machine-learned model trained to output a result of estimation of the index indicative of the hot/cold sensation by inputting an index indicative of the hot/cold sensation before correction, the sensing results of the thermal defensive action and thermal reaction may be used.

The air condition controlling unit 16 performs air-conditioning control based on the estimation result of the hot/cold sensation. At this time, the air condition controlling unit 16 controls the driving of each element of the air conditioner 1 to create an air conditioning environment comfortable for the living matter. For example, in the case of using the PMV as the index indicative of the hot/cold sensation, the air condition controlling unit 16 controls the driving of the louver, the compressor, the fan, etc. in accordance with the corrected value of the PMV, thereby controlling the temperature setting, the wind direction, the air volume, the wind velocity, the wind strength, etc.

According to the standard set by the International Organization for Standardization, a PMV range in which the predicted percentage dissatisfied (hereinafter referred to as “PPD”) is 10% or less is recommended as a comfort zone. Generally, the range of −0.5 or greater and +0.5 or smaller is known as a comfort zone of the PMV. Therefore, the living matter is presumed to feel comfortable when the value of the PMV is −0.5 or greater and +0.5 or smaller. When the value of the PMV is smaller than −0.5, the living matter is presumed to feel cold. When the value of the PMV is greater than +0.5, the living matter is presumed to feel hot.

In the case of using the PMV as the index indicative of the hot/cold sensation, when the corrected value of the PMV is −0.5 or greater and +0.5 or smaller, the air condition controlling unit 16 determines that the extracted person feels comfortable and maintains the various setting values relating to the air-conditioning control. When the corrected value of the PMV is smaller than −0.5, the air condition controlling unit 16 determines that the extracted person feels cold and, for example, increases the temperature setting. When the corrected the PMV is greater than +0.5, the air condition controlling unit 16 determines that the extracted person feels hot and, for example, decreases the temperature setting.

The air condition controlling unit 16 may control factors relating to air-conditioning control other than the wind direction, the air volume, the wind velocity, the wind strength, etc. in accordance with the corrected index indicative of the hot/cold sensation. Also, the air condition controlling unit 16 may perform controls related to the switching ON and OFF of the air conditioner 1 power supply in accordance with the corrected index indicative of the hot/cold sensation.

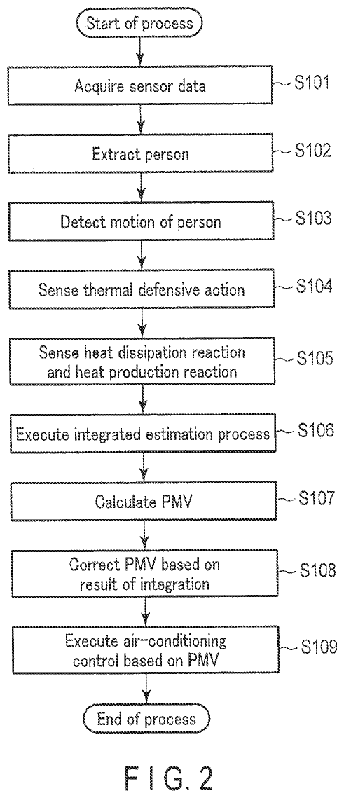

An operation of the process executed by the hot/cold sensation estimating device 10 will be explained below. FIG. 2 is a flowchart showing an example of a procedure of the hot/cold sensation estimating process. The hot/cold sensation estimating process is a process for estimating a hot/cold sensation sensed by the living matter using sensor data on vital activities of the living matter. The procedure of each process explained below is only an example and can be changed as appropriate wherever possible. Regarding the procedure explained below, steps may be omitted, replaced, and added as appropriate in accordance with the embodiment. In the following, an example in which the PMV is used as an index indicative of the hot/cold sensation and the PMV of an extracted person is calculated from the sensor data will be explained.

The hot/cold sensation estimating device 10 starts a hot/cold sensation estimating process, for example, based on the turn-on of the power supply of the air conditioner 1. When the hot/cold sensation estimating process is started, the hot/cold sensation estimating device 10 first acquires image data from each of the imaging camera 21, the thermography camera 22, and the near-infrared camera 23 through the extraction unit 11 (step S101).

Next, the hot/cold sensation estimating device 10 extracts a person as a target for estimation of the hot/cold sensation by performing a person extraction process for the image acquired from the imaging camera 21 through the extraction unit 11 (step S102). Next, the hot/cold sensation estimating device 10 performs a human tracking process through the extraction unit 11 for the image acquired from the imaging camera 21, thereby detecting a motion of the extracted person (step S103).

Next, the hot/cold sensation estimating device 10 senses the thermal defensive action of the extracted person based on a detection result of a motion by the thermal defensive action sensing unit 12 (step S104). Next, the hot/cold sensation estimating device 10 senses a heat dissipation reaction and a heat production reaction of the extracted person using the image acquired from the near-infrared camera 23 through the thermal reaction sensing unit 13 (S105).

Next, through the integrated estimation processing unit 14, the hot/cold sensation estimating device 10 calculates each of the amount of heat dissipation by the heat dissipation reaction, the amount of heat inflow by the heat production reaction, the amount of heat released from the body by the thermal defensive action, and the amount of heat produced in the body by the thermal defensive action, and executes an integrated estimation process using these calculation results (step S106).

Next, the hot/cold sensation estimating device 10 acquires an air temperature, a humidity, a wind velocity, a thermal radiation, a metabolic equivalent, and an amount of clothing from the storage medium or the image acquired from the thermography camera 22 through the hot/cold sensation estimating unit 15, and calculates a PMV using the acquired information (step S107). The PMV calculated here is an index applicable to everyone, with a personal thermoregulatory ability not reflected within. Then, the hot/cold sensation estimating device 10 corrects the calculated PMV based on the result of the integrated estimation process through the hot/cold sensation estimating unit 15 (step S108). At this time, the PMV is corrected using an integrated result based on the sensing result of the thermal defensive action and the sensing results of the heat dissipation reaction and the heat production reaction, thereby calculating an index reflecting the thermoregulatory ability of the extracted person.

Next, the hot/cold sensation estimating device 10 controls the temperature setting, the wind direction, the air volume, the wind velocity, the wind strength, etc. through the air condition controlling unit 16 in accordance with the corrected value of the PMV (S109).

Effects of the hot/cold sensation estimating device 10 according to the embodiment will be described.

The general index indicative of a hot/cold sensation is calculated in consideration of the ambient environment. For example, the air temperature, the humidity, the air volume, the atmospheric pressure, etc. used for estimation of the PMV depend on the ambient environment. On the other hand, each living matter has a thermoregulatory ability, which varies among individuals. For example, a human can make a behavioral thermoregulatory reaction with a vital reaction against the ambient temperature. In addition, a human can make an autonomic thermoregulatory reaction of voluntarily performing body temperature regulation via heat dissipation through sweating or breathing. In the general index indicative of a hot/cold sensation, the thermoregulatory ability that varies from person to person, such as the behavioral thermoregulatory reaction or the autonomic thermoregulatory reaction, is not taken into account.

The hot/cold sensation estimating device 10 according to the embodiment is configured to acquire sensor data from the sensor that senses vital activities of living matter, senses a behavioral thermoregulatory reaction and an autonomic thermoregulatory reaction of the living matter against the ambient environment, and estimates the hot/cold sensation sensed by the living matter based on the sensing result of the behavioral thermoregulatory reaction and the sensing result of the autonomic thermoregulatory reaction.

The behavioral thermoregulatory reaction is a thermal defensive reaction against heat that is generated due to an involuntary vital reaction. The hot/cold sensation estimating device 10 senses an action to raise the body temperature in cold conditions and an action to lower the body temperature in hot conditions as the behavioral thermoregulatory reactions.

The autonomic thermoregulatory reaction includes a heat dissipation reaction that dissipates heat to the ambient environment, and a heat production reaction that causes heat to flow in from the ambient environment. The hot/cold sensation estimating device 10 senses at least one of an amount of living matter perspiration, an amount of moisture on the skin surface, a frequency of breaths, or a heat amount of inhalation as the autonomic thermoregulatory reaction.

With the configuration described above, the hot/cold sensation estimating device 10 according to the embodiment can sense a thermal defensive action, and a heat dissipation reaction or a heat production reaction from the information obtained through the sensor, and estimate the ability of the living matter to self-regulate body temperature based on the sensing result. Accordingly, the hot/cold sensation in consideration of the personal thermoregulatory ability can be estimated.

Furthermore, the hot/cold sensation estimating device 10 according to the embodiment takes both a heat dissipation reaction and a heat inflow reaction into consideration, so that it can estimate the hot/cold sensation in consideration of not only the amount of moisture evaporation in hot conditions but also the heat conduction that occurs in cold conditions (in a refrigerant atmosphere).

Moreover, the hot/cold sensation estimating device 10 according to the embodiment can calculate a predictive mean vote (PMV) as an index indicative of the hot/cold sensation sensed by the living matter, and correct the calculated PMV based on the sensing result of the behavioral thermoregulatory reaction and the sensing result of the autonomic thermoregulatory reaction.

With the configuration described above, the hot/cold sensation estimating device 10 according to the embodiment corrects the index indicative of the hot/cold sensation applicable to everyone, such as the PMV, in accordance with the personal thermoregulatory ability, so that a personally specialized hot/cold sensation can be estimated. In other words, the hot/cold sensation is estimated by using information relating to the ability of the living matter to self-regulate body temperature in addition to the information on the ambient environment, so that the hot/cold sensation in consideration of the personal thermoregulatory ability can be estimated.

The sensor is a non-contact sensor including at least one of an imaging camera, a depth camera, a thermography camera, a near-infrared camera, or a radar. The non-contact sensor is used as the sensor that senses vital activities of living matter, so that the thermoregulatory ability can be estimated without attaching a sensor to the extracted person.

The air conditioner 1 according to the embodiment includes the sensor and the hot/cold sensation estimating device 10, and can perform air-conditioning control based on the estimation result of the hot/cold sensation. With the configuration described above, the air-conditioning control is performed on the basis of the estimation result of the hot/cold sensation in consideration of the personal thermoregulatory ability, thereby realizing individually distributed air conditioning, so that more precise air-conditioning control can be achieved.

### First Modification of First Embodiment

A first modification of the first embodiment will be described. In the modification, the configuration of the first embodiment is modified as follows: The descriptions of the same configurations, operations, and effects as those of the first embodiment are omitted. An air conditioner 1 including a hot/cold sensation estimating device 10 according to the modification estimates a hot/cold sensation which living matter senses based on a result of thermoregulatory ability estimation in addition to a behavioral thermoregulatory reaction and an autonomic thermoregulatory reaction.

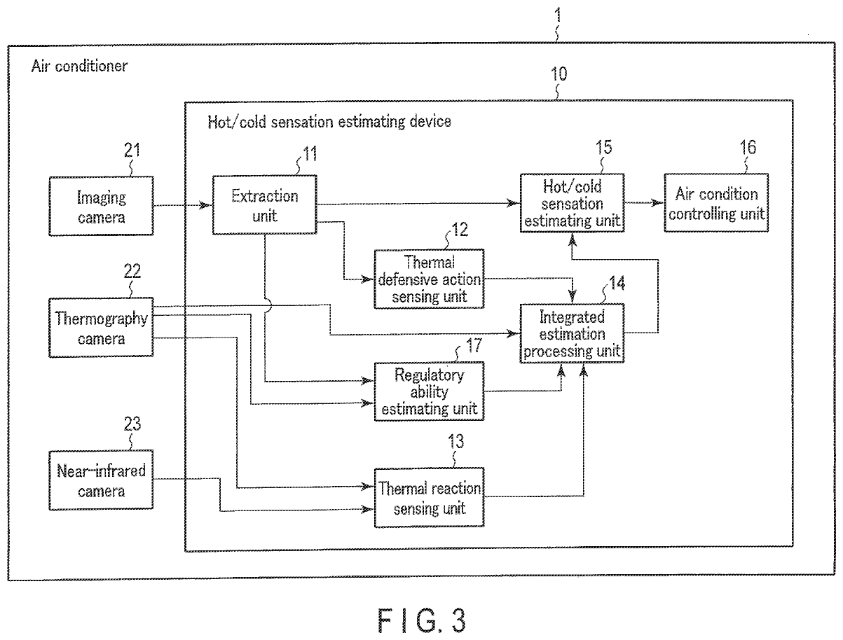

FIG. 3 is a diagram showing the air conditioner 1 including the hot/cold sensation estimating device 10 according to the modification. As shown in FIG. 3, the processing circuit of the hot/cold sensation estimating device 10 further executes a function of a regulatory ability estimating unit 17. The processing circuit that realizes the regulatory ability estimating unit 17 corresponds to a part of sensing units.

The thermal defensive estimate unit 17 senses a thermoregulatory ability of living matter based on sensor data. For example, the regulatory ability estimating unit 17 calculates an amount of change in body temperature per unit time on the skin surface of living matter extracted by the extraction unit 11 and an amount of change in ambient temperature around the living matter per unit time, using a plurality of thermography images sequentially acquired over time. Then, the regulatory ability estimating unit 17 estimates an index relating to the thermoregulatory ability of the living matter based on the calculated amounts of change in both body temperature and ambient temperature. The index relating to the thermoregulatory ability is, for example, a rate of change of the amount of change in living matter body temperature relative to the amount of change in ambient temperature between any two points in time. The amount of change in ambient temperature may be calculated based on data acquired through an environment sensor, such as a thermometer different from the thermography camera 22. To improve the accuracy of thermoregulatory ability estimation, the ambient temperature may be actively changed by air-conditioning control.

The integrated estimation processing unit 14 performs an integrated estimation process with respect to a sensing result of a thermal defensive action, a sensing result of a heat dissipation reaction, and a result of thermoregulatory ability estimation. Specifically, the integrated estimation processing unit 14 applies the index indicative of the thermoregulatory ability to a given integration algorithm, in addition to the calculated amounts of heat dissipation, heat inflow, heat released from the body, and heat produced in the body, thereby integrating the sensing result of the thermal defensive action, the sensing result of the heat dissipation reaction, and the result of thermoregulatory ability estimation.

The hot/cold sensation estimating unit 15 estimates a hot/cold sensation sensed by the living matter based on the sensing result of the behavioral thermoregulatory reaction, the sensing result of the autonomic thermoregulatory reaction, and the result of thermoregulatory ability estimation. When the PMV is used as an index indicative of a hot/cold sensation, the hot/cold sensation estimating unit 15 calculates the PMV as an index indicative of the hot/cold sensation sensed by the living matter, and corrects the calculated value of the PMV based on an integrated result of the sensing result of the thermal defensive action, the sensing result of the heat dissipation reaction, and the result of thermoregulatory ability estimation.

For example, when the value of the index relating to the thermoregulatory ability is equal to or greater than a predetermined value, the hot/cold sensation estimating unit 15 determines that the living matter has a high thermoregulatory ability. In this case, the hot/cold sensation estimating unit 15 increases the amount of PMV to be corrected based on the sensing result of the thermal defensive action and the sensing result of the thermal reaction in accordance with the value of the index relating to the thermoregulatory ability. On the other hand, when the value of the index relating to the thermoregulatory ability is smaller than the predetermined value, the hot/cold sensation estimating unit 15 determines that the living matter has a low thermoregulatory ability. In this case, the hot/cold sensation estimating unit 15 decreases the amount of PMV to be corrected based on the sensing result of the thermal defensive action and the sensing result of the thermal reaction in accordance with the value of the index relating to the thermoregulatory ability.

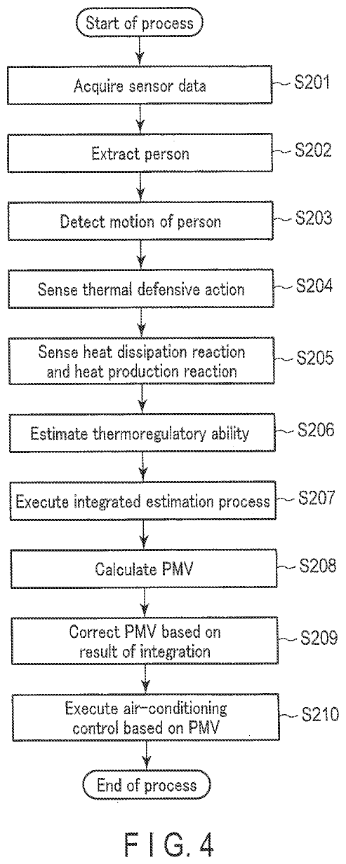

An operation of the hot/cold sensation estimating process executed by the hot/cold sensation estimating device 10 according to the embodiment will be explained below. FIG. 4 is a flowchart showing an example of a procedure of the hot/cold sensation estimating process according to the embodiment. As explained with reference to FIG. 2, an example in which the PMV is used as an index indicative of the hot/cold sensation and the PMV of an extracted person is calculated from the sensor data will be explained. The processes in steps S201-S205 and step S210 are respectively the same as the processes in steps S101-S105 and step S109 shown in FIG. 2, and the explanations thereof will be omitted.

After sensing a thermal defensive action, a heat dissipation reaction, and a heat production reaction of a person extracted through the processes in steps S201-S205, the hot/cold sensation estimating device 10 calculates an index relating to a thermoregulatory ability of the extracted person through the regulatory ability estimating unit 17 using a plurality of images acquired from the thermography camera 22 (step S206).

Next, through the integrated estimation processing unit 14, the hot/cold sensation estimating device 10 calculates each of the amount of heat dissipation by the heat dissipation reaction, the amount of heat inflow by the heat production reaction, the amount of heat released from the body by the thermal defensive action, the amount of heat produced in the body by the thermal defensive action, and the index relating to the thermoregulatory ability, and executes an integrated estimation process using these calculation results (step S207).

Through the hot/cold sensation estimating unit 15, the hot/cold sensation estimating device 10 calculates the PMV of the extracted person (step S208) and corrects the calculated PMV based on the result of the integrated estimation process (step S209). At this time, the PMV is calculated, reflecting the result of thermoregulatory ability estimation in addition to the sensing result of the thermal defensive action and the sensing results of the heat dissipation reaction and the heat production reaction.

Hereinafter, effects of the hot/cold sensation estimating device 10 according to the modification will be described.

The hot/cold sensation estimating device 10 according to the modification can estimate a thermoregulatory ability of the living matter based on the sensor data, and estimate a hot/cold sensation sensed by the living matter based on the sensing result of the behavioral thermoregulatory reaction, the sensing result of the autonomic thermoregulatory reaction, and the result of thermoregulatory ability estimation.

Due to the configuration described above, the hot/cold sensation estimating device 10 according to the modification can estimate a personal thermoregulatory ability based on an actual change in body temperature over time. Accordingly, it is possible to estimate a more precise hot/cold sensation sensed by the living matter in consideration of factors other than the behavioral thermoregulatory reaction and the autonomic thermoregulatory reaction that influence thermoregulatory ability.

The thermoregulatory ability may be estimated in consideration of the time when the image for use in estimation of the thermoregulatory ability was captured. Generally, it is known that the body temperature is higher in the evening or night, for example, 14:00 to 18:00, than in the morning, for example, 3:00 to 7:00. Therefore, for example, when the thermoregulatory ability in the night is estimated by using a thermography image captured in the morning, it is determined that the actual thermoregulatory ability is higher than the result of thermoregulatory ability estimation, and the thermoregulatory ability estimation result is corrected.

### Another Modification of First Embodiment

Alternatively, profile data of the living matter as a target for estimation of the hot/cold sensation may be taken into consideration. For example, the user registers beforehand profile data relating to a person who may use the room where the air conditioner 1 is located. The air conditioner 1 extracts the person as a target for estimation of the hot/cold sensation, and thereafter specifies the extracted person based on the registered profile data. The profile data may be input through the controller of the air conditioner 1, or through an information terminal device connected to the air conditioner 1 via a network.

The profile data is information including, for example, age, ethnicity, sex, height, weight, cognitive capacity, health history, sensitivity to heat, sensitivity to cold, a body part that easily cools, hometown, parents' hometown, sweat gland, Eccrine sweat glands, etc. Health history information includes information on a brain hemorrhage, brain infarction, high blood pressure, lung disease, diabetes, anemia, kidney disease, mental illness, thyroid disease, heat stroke, smoking habits, etc.

For example, the air conditioner 1 may correct the estimation result of the hot/cold sensation in consideration of the deterioration of the hot/cold sensation if the person has a heart or lung disease. The air conditioner 1 may also correct the estimation result of the thermoregulatory ability estimated by the regulatory ability estimating unit 17 in consideration of the thermoregulatory ability deterioration if the person is a child, a baby, elderly, etc. The information on ethnicity, sweat gland, Eccrine sweat glands, or the like is used to estimate the number of effective pores. The information on the hometown of the person and their parents' hometown is used to estimate the resistance to hotness and coldness, the number of pores based on information relating to climate, for example, the atmospheric temperature or the humidity of the region. For example, the air conditioner 1 may correct the result of the thermoregulatory ability after factoring in that higher numbers of effective pores create higher thermoregulatory ability. Furthermore, using the information on the body part that cools easily, the air conditioner 1 may control the wind direction or the like to effectively apply the wind to the aforementioned body part and thereby cool the entire body.

If a plurality of persons are extracted from the sensor data as targets of estimation of the hot/cold sensation, the hot/cold sensation of each of the extracted persons may be estimated, so that the air-conditioning control can be performed to make an air condition environment comfortable for all persons. Furthermore, priorities of family members may be set in advance, and if a plurality of persons among family members are specified, the air-conditioning control may be adapted to the person of the highest priority.

The hot/cold sensation estimating process may be performed while the power supply of the air conditioner 1 is OFF. For example, the specification of a person extracted from an image acquired from the imaging camera 21 and estimation of the hot/cold sensation of the specified person may be performed over time, and in the case of, for example, an elderly adult with a low heat comfort level, the power supply of the air conditioner 1 may be automatically turned from OFF to ON. In this manner, an elderly adult living alone can be prevented from suffering heat stroke.

Furthermore, whether the living matter as a target for estimation of the hot/cold sensation puts on or takes off clothes may be detected based on the image acquired from the imaging camera 21 or the thermography camera 22, and the estimation result of the hot/cold sensation may be corrected in consideration of improvements in comfort by the putting on or removal of clothes.

Moreover, the thermography camera 22 may detect temperatures of the respective body part, such as a peripheral body part and a center of the body, and calculate an error between the detected temperature and a preset target temperature for each body part, so that the air-conditioning control may be performed to minimize the sum of the errors or the sum of calculated errors with weights.

Furthermore, any stress that the living matter as a target for estimation of the hot/cold sensation feels may be sensed using the face image acquired from the imaging camera 21, and the estimation result of the hot/cold sensation may be corrected in consideration of the stress sensing result.

### Second Embodiment

A second embodiment will be described. In the embodiment, the configuration of the first embodiment is modified as follows: The descriptions of the same configurations, operations, and effects as those of the first embodiment are omitted. An air conditioner 1 including a hot/cold sensation estimating device 10 of the embodiment is located in, for example, an office, an exhibition hall, a conference room, a meeting space, etc. The hot/cold sensation estimating device 10 estimates an intellectual productivity of living matter as a target for estimation of a hot/cold sensation from a camera image and a microphone voice, and corrects the hot/cold sensation based on the estimation result, thereby performing an air-conditioning control.

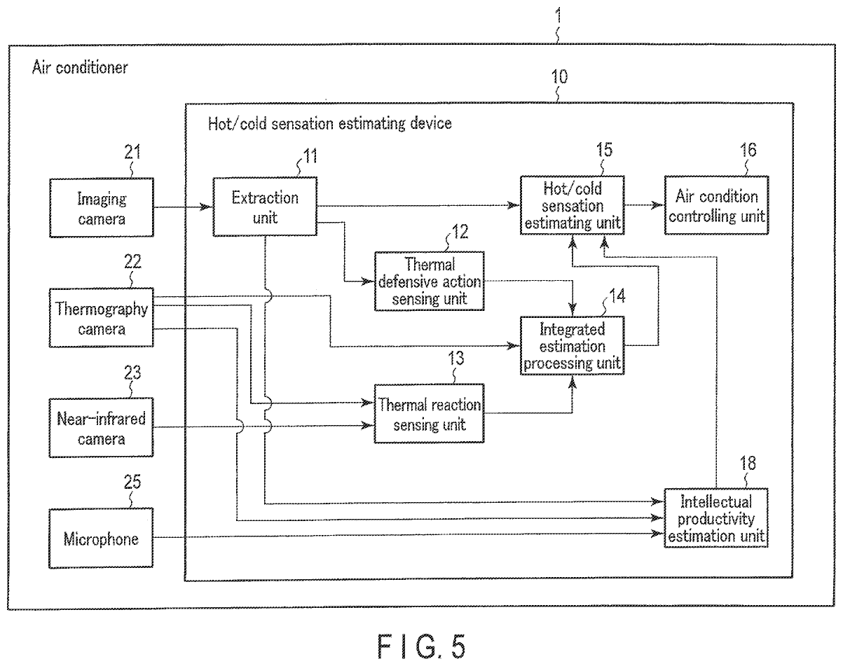

FIG. 5 is a diagram showing a configuration of the air conditioner 1 including the hot/cold sensation estimating device 10 according to the embodiment. As shown in FIG. 5, the air conditioner 1 further includes a microphone 25. The microphone 25 is a sound collector which detects a voice of a person as a target for estimation of a hot/cold sensation. Voice data acquired through the microphone 25 is output to an intellectual productivity estimation unit 18. The microphone 25 is an example of a sensor. The voice data is an example of sensor data.

A processing circuit of the hot/cold sensation estimating device 10 further executes a function of the intellectual productivity estimation unit 18. The processing circuit that realizes the intellectual productivity estimation unit 18 corresponds to a part of estimation units.

The intellectual productivity estimation unit 18 estimates a person's intellectual productivity as a target for estimation of the hot/cold sensation based on the sensor data. The person as a target for estimation of the hot/cold sensation is, for example, a client who is given an explanation about something. The intellectual productivity includes production efficiency and creativity. For example, the intellectual productivity estimation unit 18 detects information on a face and a head, a pose, and on an amount of limb motion from image data, and detects an amount and content of speech of the extracted person based on the voice data acquired through the microphone 25. The intellectual productivity estimation unit 18 estimates an intellectual productivity of the person as a target for estimation of the hot/cold sensation using the detection results.

The information on a face and a head includes, for sample, a facial expression, a surface temperature of the skin of the face, an instantaneous stress value, a motion of the head, or the like. The facial expression is, for example, a thinking expression, a sleepy expression, or the like. The thinking expression, the sleepy expression, and the instantaneous stress value are detected, for example, based on an image acquired from the imaging camera 21. The surface temperature of facial skin is detected, for example, based on a thermography image. The motion of the head includes, for example, a rate of lifting the face, a frequency of nodding or yawning, a neck motion or inclination, or the like. The head motion is detected, for example, based on the image acquired from the imaging camera 21.

The information on the pose includes, for example, a bodily motion, an upper body pose, a passive pose, a pose not associated with laziness, or the like. The pose information is detected, for example, based on the image acquired from the imaging camera 21.

The information on an amount of limb motion includes, for example, a hand motion, an action of pointing an object, such as an exhibited object, an action of touching an object, such as an exhibited object, a leg motion, a leg vibration frequency, or the like. The information on an amount of limb motion is detected, for example, based on the image acquired from the imaging camera 21 or the thermography image.

The amount of speech is an amount of speech made by the person as a target for estimation of the hot/cold sensation. The content of speech is a content of speech made by person as a target for estimation of the hot/cold sensation. The amount and content of speech is detected via application of a known speaker classification algorithm or speaker recognition algorithm to the voice data acquired through the microphone 25.

The hot/cold sensation estimating unit 15 estimates a hot/cold sensation sensed by the living matter based on the result of intellectual productivity estimation. In the case of using the PMV as the index indicative of the hot/cold sensation, the hot/cold sensation estimating unit 15 further corrects the value of the corrected PMV based on the result of intellectual productivity estimation.

For example, if the intellectual productivity of the extracted person is estimated to be low, the hot/cold sensation estimating unit 15 determines that the heat comfort of the extracted person is low and corrects the value of the PMV to be apart from the comfort zone. On the other hand, if the intellectual productivity of the extracted person is estimated to be high, the hot/cold sensation estimating unit 15 determines that the heat comfort of the extracted person is sufficiently high, and corrects the value of the PMV to approach the comfort zone or maintains the value of the PMV.

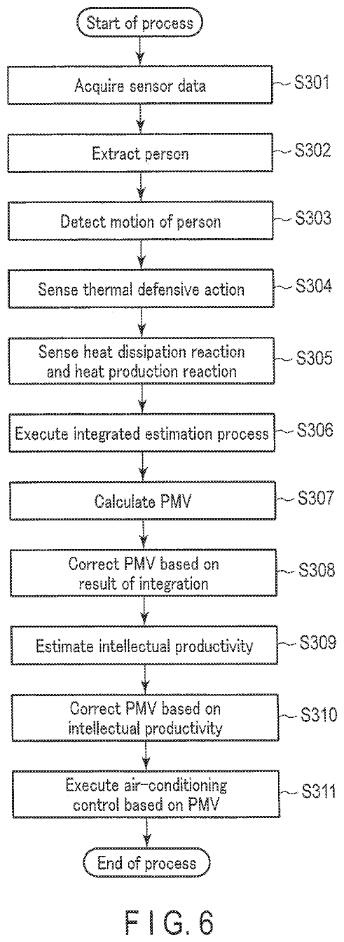

An operation of the hot/cold sensation estimating process executed by the hot/cold sensation estimating device 10 according to the embodiment will be explained below. FIG. 6 is a flowchart showing an example of a procedure of the hot/cold sensation estimating process according to the embodiment. As explained with reference to FIG. 2, an example in which the PMV is used as an index indicative of the hot/cold sensation and the PMV of an extracted person is calculated from the sensor data will be explained. The processes in steps S301-S308 and step S311 are respectively the same as those in steps S101-S109 shown in FIG. 2, and the explanations thereof will be omitted.

The hot/cold sensation estimating device 10 corrects the calculated PMV based on the result of the integrated estimation process through the process in step S301-S308, and thereafter estimates an intellectual productivity of the extracted person based on the image data and the voice data through the intellectual productivity estimation unit 18 (step S309).

Next, the hot/cold sensation estimating device 10 further corrects the corrected PMV based on the result of intellectual productivity estimation through the hot/cold sensation estimating unit 15 (step S310).

Hereinafter, effects of the hot/cold sensation estimating device 10 according to the embodiment will be described.

The hot/cold sensation estimating device 10 according to the embodiment can estimate an intellectual productivity of the living matter based on the sensor data, and estimate a hot/cold sensation sensed by the living matter based on the result of intellectual productivity estimation. Specifically, after calculating the index indicative of heat comfort, such as the PMV, the calculated index is corrected in accordance with the estimated intellectual productivity, so that the estimation accuracy of the hot/cold sensation sensed by the living matter can be improved. In addition, through performing the air-conditioning control in accordance with the correction result, an air condition environment that improves intellectual productivity can be created.

For example, it is known that the intellectual productivity of office staff is lowered in either hot or cold conditions. According to the hot/cold sensation estimating device 10 of the embodiment, the condition of the office staff is analyzed from the camera image and the microphone voice, so that the presence or absence of new ideas generated by those staff can be ascertained by estimating intellectual productivity of the office staff. Furthermore, the condition of the client who is given an explanation by exhibition staff is analyzed from the camera image and the microphone voice, so that it can be ascertained whether the client has received a positive or negative impression by estimating intellectual productivity of the office staff. In addition, through the intellectual productivity estimation of persons set to join a meeting, whether or not the meeting should be established and successful can be ascertained.

The hot/cold sensation estimating device 10 is not necessarily disposed in the air conditioner. For example, the hot/cold sensation estimating unit 10 may be mounted on a system that notifies the exhibition staff of the estimation result of the hot/cold sensation. In this case, the hot/cold sensation estimating device 10 is located in the exhibition hall together with the imaging camera and the microphone. The hot/cold sensation estimating device 10 includes a loud speaker that notifies the staff of the estimation result of the hot/cold sensation. The hot/cold sensation estimating device 10 may include a display configured to display the estimation result of the hot/cold sensation. The hot/cold sensation estimating device 10 estimates the hot/cold sensation of the client given an explanation from the relevant staff through the camera image and the microphone voice, and notifies the staff of the estimation result of the hot/cold sensation. The staff can ascertain the hot/cold sensation sensed by the client by ascertaining the notification and change their client care actions accordingly.

Thus, according to the embodiments, it is possible to provide a hot/cold sensation estimating device, method, and program for estimating hot/cold sensation in consideration of the thermoregulatory ability of living matter.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A hot/cold sensation estimating device comprising a processing circuit configured to:
sense a behavioral thermoregulatory reaction and an autonomic thermoregulatory reaction of living matter against an ambient environment based on sensor data acquired through a sensor for sensing vital activities of the living matter; and
estimate a hot/cold sensation sensed by the living matter based on a sensing result of the behavioral thermoregulatory reaction and a sensing result of the autonomic thermoregulatory reaction.

2. The hot/cold sensation estimating device according to claim 1, wherein the behavioral thermoregulatory reaction is a thermal defensive reaction resulting from a vital reaction.

3. The hot/cold sensation estimating device according to claim 2, wherein the behavioral thermoregulatory reaction includes an action to raise a body temperature and an action to lower the body temperature.

4. The hot/cold sensation estimating device according to claim 1, wherein the autonomic thermoregulatory reaction includes a heat dissipation reaction that dissipates heat to the ambient environment, and a heat production reaction that causes heat to flow in from the ambient environment.

5. The hot/cold sensation estimating device according to claim 4, wherein the processing circuit is configured to sense at least one of: an amount of living matter perspiration, an amount of moisture on a skin surface, a frequency of breaths, a heat amount of exhalation, or a heat amount of inhalation.

6. The hot/cold sensation estimating device according to claim 1, wherein the processing circuit is configured to calculate a predictive mean vote as an index indicative of the hot/cold sensation sensed by the living matter, and correct the calculated predictive mean vote based on the sensing result of the behavioral thermoregulatory reaction and the sensing result of the autonomic thermoregulatory reaction.

7. The hot/cold sensation estimating device according to claim 1, wherein the sensor is a non-contact sensor including at least one of an imaging camera, a depth camera, a thermography camera, a near-infrared camera, a radar, or a ToF sensor.

8. The hot/cold sensation estimating device according to claim 1, wherein the processing circuit is configured to:
estimate a thermoregulatory ability of the living matter based on the sensor data; and
estimate the hot/cold sensation sensed by the living matter based on the sensing result of the behavioral thermoregulatory reaction, the sensing result of the autonomic thermoregulatory reaction, and a result of thermoregulatory ability estimation.

9. The hot/cold sensation estimating device according to claim 8, wherein the processing circuit is configured to calculate a rate of change of an amount of change in body temperature of the living matter relative to an amount of change in ambient temperature, and estimate the thermoregulatory ability based on a calculation result.

10. The hot/cold sensation estimating device according to claim 1, wherein the hot/cold sensation estimating device is provided in an air conditioner,
and the processing circuit is configured to perform an air-conditioning control of the air conditioner based on an estimation result of the hot/cold sensation.

11. The hot/cold sensation estimating device according to claim 1, wherein the processing circuit is configured to estimate an intellectual productivity of the living matter based on the sensor data, and estimate the hot/cold sensation sensed by the living matter based on a result of intellectual productivity estimation.

12. A hot/cold sensation estimating method comprising:
sensing a behavioral thermoregulatory reaction and an autonomic thermoregulatory reaction of living matter against an ambient environment based on sensor data acquired through a sensor for sensing vital activities of the living matter; and
estimating a hot/cold sensation sensed by the living matter based on a sensing result of the behavioral thermoregulatory reaction and a sensing result of the autonomic thermoregulatory reaction.

13. A non-transitory computer-readable storage medium storing a program for causing a computer to execute:
a function of sensing a behavioral thermoregulatory reaction and an autonomic thermoregulatory reaction of living matter against an ambient environment based on sensor data acquired through a sensor for sensing vital activities of the living matter; and
a function of estimating a hot/cold sensation sensed by the living matter based on a sensing result of the behavioral thermoregulatory reaction and a sensing result of the autonomic thermoregulatory reaction.
